(19) United States
(12) Patent Application Publication
(10) Pub. No.: US 2018/0177849 A1
(43) Pub. Date: Jun. 28, 2018

(54) Therapeutic Uses of Elsiglutide

(71) Applicant: HELSINN HEALTHCARE SA, LUGANO/PAZZALLO (CH)

(72) Inventors: Claudio Pietra, Como (IT); Emanuela Lovati, Mendrisio (CH); Youcef M. Rustum, Rockville, MD (US)

(21) Appl. No.: 15/726,439

(22) Filed: Oct. 6, 2017

Related U.S. Application Data
(63) Continuation of application No. 15/266,907, filed on Sep. 15, 2016, now abandoned.
(60) Provisional application No. 62/219,833, filed on Sep. 17, 2015.

(51) Int. Cl. A61K 38/26; A61K 45/06; A61K 31/4745

(52) U.S. Cl. CPC A61K 38/26; A61K 45/06; A61K 31/4745

(57) ABSTRACT

The invention relates to therapeutic uses of elsiglutide, particularly for protecting and stimulating bone marrow and peripheral blood cell type activity or immune-response in patients compromised due to the administration of chemotherapeutic agents. The invention further provides uses of elsiglutide to enhance the antitumor activity of cytotoxic chemotherapeutic agents and target specific biological agents.

### FIELD OF THE INVENTION

The invention relates to therapeutic uses of elsiglutide, particularly to protect against acute and chronic toxicity and to stimulate hematologic activity, including in bone marrow, peripheral blood cell types, and the immune system in patients receiving chemotherapeutic agents. The invention further provides uses of elsiglutide to enhance the therapeutic efficacy of chemotherapeutic agents.

### BACKGROUND OF THE INVENTION

Cytotoxic drugs used in chemotherapy produce many negative side effects. Myelosuppression, a condition in which reproduction of cells in the bone marrow is suppressed, is one of the most impactful and harmful side-effects. Myelosuppression causes anemia (low red blood cell counts), neutropenia (low neutrophils counts), leucopenia (low white blood cell counts), and thrombocytopenia (low platelet counts). Myelosuppression can also be felt as fatigue due to anemia, increased infections due to neutropenia, and bruising and bleeding due to thrombocytopenia.

Glucagon-like-peptide-2 (GLP-2) is a 33-amino-acid peptide released from the post-translational processing of proglucagon in the enteroendocrine L cells of the intestine and in specific regions of the brainstem. It is co-secreted together with glucagon-like peptide 1 (GLP-1), oxyntomodulin and glicentin, in response to nutrient ingestion. GLP-2 induces significant growth of the small intestinal mucosal epithelium via the stimulation of stem cell proliferation in the crypts and inhibition of apoptosis on the villi (Drucker et al. Proc Natl Acad Sci USA. 1996, 93:7911-6). GLP-2 also inhibits gastric emptying and gastric acid secretion (Wojdemann et al. J Clin Endocrinol Metab. 1999, 84:2513-7), enhances intestinal barrier function (Benjamin et al. Gut. 2000, 47:112-9), stimulates intestinal hexose transport via the upregulation of glucose transporters (Cheeseman, Am J Physiol. 1997, R1965-71), and increases intestinal blood flow (Guan et al. Gastroenterology. 2003, 125, 136-47).

The benefits of GLP-2 in the small intestine have raised much interest in the use of GLP-2 in the treatment of intestinal disease or injury (Sinclair and Drucker, Physiology 2005: 357-65). Furthermore GLP-2 has been shown to prevent or reduce mucosal epithelial damage in a number of preclinical models of gut injury, including chemotherapy-induced mucositis, ischemia-reperfusion injury, dextran sulfate-induced colitis and genetic models of inflammatory bowel disease (Sinclair and Drucker, Physiology 2005:357-65).

GLP-2 is secreted as a 33 amino acid peptide having the sequence HADGSFSDEMNTILDNLAARDFINWLIQTKITD (SEQ ID NO: 2). It is rapidly cleaved at the Alanine (A) in position 2 of the N-terminus to the inactive human GLP-2 (3-33) by the enzyme dipeptidyl peptidase-4 (DPP IV). This rapid enzymatic degradation of GLP-2(1-33), in addition to renal clearance results in a half-life of about 7 minutes for the peptide (Tavares et al., Am. J. Physiol. Endocrinol. Metab. 278:E134-E139, 2000).

U.S. Pat. Nos. 7,745,403 and 7,563,770 disclose GLP-2 analogues which comprise one of more substitutions as compared to wild-type GLP-2. One of the described GLP-2 analogues is ZP1846 (elsiglutide). A comparison of the sequences of GLP-2 and elsiglutide is provided below:

elsiglutide:
(SEQ ID NO: 1)
HGEGSFSSELSTILDALAARDFIAWLIATKITDKKKKKK
GLP-2:
(SEQ ID NO: 2)
HADGSFSDEMNTILDNLAARDFINWLIQTKITD.

U.S. Pat. Nos. 7,745,403 and 7,563,770 propose the use of GLP-2 analogues, including elsiglutide, for preventing or ameliorating side effects of chemotherapy, including chemotherapy-induced diarrhea (CID). GLP-2 analogues appear to act in CID by inhibiting enterocyte and crypt cell apoptosis and increasing crypt cell proliferation, thus providing new cells to replace the damaged intestinal epithelium following chemotherapy.

A planned experimental trial of elsiglutide was reported on clinicaltrials.gov sometime around Feb. 21, 2012. The official title of the trial was Phase II, Double-blind, Randomized, Two-stage, Placebo-controlled Proof of Concept Study in Colorectal Cancer Patients Receiving 5-FU Based Chemotherapy to Assess the Efficacy of Elsiglutide (ZP1846) Administered s.c. in the Prevention of Chemotherapy Induced Diarrhea (CID). Clinicaltrials.gov reports the following brief summary of the study: The main objective of this study will be to obtain data on the efficacy of elsiglutide in preventing Chemotherapy Induced Diarrhea (CID) in patients with colorectal cancer receiving 5-FU based chemotherapy (FOLFOX4 or FOLFIRI regimen) in comparison to placebo.

### SUMMARY OF THE INVENTION

The present invention is based on the unexpected discovery that myelosuppression and immuno-compromise caused by cytotoxic agents can be reversed by administering the GLP-2 analog elsiglutide. Therefore, in a first principal embodiment the invention provides a method of improving the immunological status of a subject immune-compromised as a consequence of cytotoxic therapy comprising administering to said subject an elsiglutide regimen before, during or after administering a cycle of said cytotoxic therapy to said subject.

In a second principal embodiment the invention provides a method of increasing bone marrow activity in a subject suffering myelosuppression as a consequence of cytotoxic therapy comprising administering to said subject an elsiglutide regimen before, during or after administering a cycle of said cytotoxic therapy to said subject.

The invention also related to the unexpected discovery that elsiglutide can enhance the effect of cytotoxic agents. Thus, in a third principal embodiment the invention provides a method of enhancing the effectiveness of cytotoxic therapy in a subject receiving cytotoxic therapy for the treatment of cancer comprising administering to said subject an elsiglutide regimen before, during or after administering a cycle of said cytotoxic therapy to said subject.

The foregoing and other objects, features and advantages of the invention will be apparent from the following more particular description of preferred embodiments of the invention.

### BRIEF DESCRIPTION OF THE FIGURES

This patent application file contains at least one drawing executed in color. Copies of this patent application with color drawing(s) will be provided by the Office upon request and payment of the necessary fee.

FIG. 1 demonstrates changes in average numbers of major peripheral blood cell types (white blood cells, red blood cells and platelets) in various groups of animal studies, as described in greater detail in Example 2.

### DETAILED DESCRIPTION OF THE INVENTION

The present invention may be understood more readily by reference to the following detailed description of preferred embodiments of the invention and the Examples included therein.

### Definitions and Use of Terms

As used in the specification and claims, the singular forms a, an, and the include plural references unless the context clearly dictates otherwise. For example, the term “a pharmaceutical excipient” refers to one or more pharmaceutical excipients for use in the presently disclosed formulations and methods.

When ranges are given by specifying the lower end of a range separately from the upper end of the range, it will be understood that the range can be defined by selectively combining any one of the lower end variables with any one of the upper end variables that is mathematically possible.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All references cited herein, including patents, patent applications, and published patent applications, are hereby incorporated by reference in their entireties, whether or not each is further individually incorporated by reference.

The term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

When a peptide active ingredient is referred to herein in its native form, it will be understood to include all pharmaceutically acceptable salts thereof. Thus, references to elsiglutide include elsiglutide hydrochloride, and other pharmaceutically acceptable salts of elsiglutide.

As used herein, the term “elsiglutide” or “ZP1846” refers to a GLP-2 peptide analog having amino acid SEQ ID NO: 1. The term also encompasses peptides provided in the form of a salt. Salts include pharmaceutically acceptable salts such as, e.g., acid addition salts and basic salts. Non-limiting examples of acid addition salts include hydrochloride salts, citrate salts and acetate salts. Non-limiting examples of basic salts include salts where the cation is selected from alkali metals, such as sodium and potassium, alkaline earth metals, such as calcium, and ammonium ions ⁺N(R³)₃(R⁴), where R³ and R⁴ independently designates optionally substituted C₁₋₆-alkyl, optionally substituted C₂₋₆-alkenyl, optionally substituted aryl, or optionally substituted heteroaryl. Other examples of pharmaceutically acceptable salts are described in “Remington's Pharmaceutical Sciences”, 17th edition. Ed. Alfonso R. Gennaro (Ed.), Mark Publishing Company, Easton, Pa., U.S.A., 1985 and more recent editions, and in the Encyclopaedia of Pharmaceutical Technology.

The terms “chemotherapy” and “cytotoxic therapy” are used interchangeably herein to refer to the administration of chemical agents for the purposes of killing or inhibiting replication of cells in a mammal, typically for the treatment of cancer. The terms “anti-cancer agent” and “chemotherapeutic agent” are used herein to refer to any chemical compound which is used to treat cancer. Chemotherapeutic agents are well known in the art (see, e.g., Gilman A. G., et al., The Pharmacological Basis of Therapeutics, 8th Ed., Sec 12:1202-1263 (1990)). Specific non-limiting examples of chemotherapeutic agents are provided throughout the specification and include, for example, FOLFOX (a chemotherapy regimen for treatment of colorectal cancer, which comprises administration of folinic acid (leucovorin), fluorouracil (5-FU), and oxaliplatin) and FOLFIRI (a chemotherapy regimen for treatment of colorectal cancer, which comprises administration of folinic acid (leucovorin), fluorouracil (5-FU), and irinotecan), as well as administration of targeted monoclonal antibody therapy (e.g., bevacizumab, cetuximab, or panitumumab) alone or in combination with chemotherapeutic agents.

The terms “chemotherapy cycle” and “cycle of cytotoxic therapy” are used herein to refer to a period of time between the initial administration of an anti-cancer agent and its repeat administration. For example, the cycle of the FOLFOX4 chemotherapy includes 14 days, wherein anti-cancer agents are administered only for the first 2 days of the cycle as follows: Day 1: oxaliplatin 85 mg/m² IV infusion and leucovorin 200 mg/m² IV infusion both given over 120 minutes at the same time in separate bags, followed by 5-FU 400 mg/m² IV bolus given over 2-4 minutes, followed by 5-FU 600 mg/m² IV infusion as a 22-hour continuous infusion; Day 2: leucovorin 200 mg/m² IV infusion, followed by 5-FU 400 mg/m² IV bolus given over 2-4 minutes, followed by 5-FU 600 mg/m² IV infusion as a 22-hour continuous infusion. Similarly, the cycle of the FOLFIRI chemotherapy discussed in the Examples section, below, includes 14 days, wherein anti-cancer agents are administered only for the first 2 days of the cycle as follows: irinotecan (180 mg/m² IV over 90 minutes) concurrently with folinic acid (400 mg/m² [or 2×250 mg/m²] IV over 120 minutes), followed by fluorouracil (400-500 mg/m² IV bolus) then fluorouracil (2400-3000 mg/m² intravenous infusion over 46 hours). Bevacizumab is usually given intravenously every 14 days, although the frequency can be dose dependent (for example 5 mg/kg by intravenous infusion every two weeks or 7.5 mg/kg by intravenous infusion every three weeks). In colon cancer, it is given in combination with the chemotherapy drug 5-FU (5-fluorouracil), leucovorin, and oxaliplatin or irinotecan. One recommended dose and schedule for cetuximab is 400 mg/m² administered intravenously as a 120-minute infusion as an initial dose, followed by 250 mg/m² infused over 30 minutes weekly, preferably in combination with FOLFIRI.

The terms “co-administered” and “co-administration” broadly refer to administration of two or more components, compounds or compositions (e.g., a chemotherapeutic agent and elsiglutide), wherein said components, compounds or compositions can be administered either simultaneously (in one composition) or in two or more separate compositions.

In the context of the present invention insofar as it relates to any of the disease conditions recited herein, the terms “treat” and “treatment” and the like mean to relieve or alleviate at least one symptom associated with such condition, or to slow or reverse the progression of such condition. Within the meaning of the present invention, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis, etc.

As used herein the term “therapeutically effective” applied to a dose or amount refers to that quantity of a compound or pharmaceutical composition that is sufficient to result in a desired activity upon administration to a subject in need thereof. Within the context of the present invention, when the term “therapeutically effective” is used in connection with elsiglutide, it refers to an amount of elsiglutide or a pharmaceutical composition containing elsiglutide that is effective to ameliorate or prevent side effects of cancer chemotherapy or to increase the efficacy of cancer chemotherapy. Note that when a combination of active ingredients is administered (e.g., a combination of elsiglutide and another compound effective for ameliorating or preventing side effects of cancer chemotherapy) the effective amount of the combination may or may not include amounts of each ingredient that would have been effective if administered individually.

The phrase “pharmaceutically acceptable”, as used in connection with compositions of the invention, refers to molecular entities and other ingredients of such compositions that are physiologically tolerable and do not typically produce untoward reactions when administered to a subject (e.g., a mammal such as a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

As used herein, the term “subject” refers to any mammal. In a preferred embodiment, the subject is human.

### Therapeutic Methods of the Invention

In a first principal embodiment the invention provides a method of improving the immunological status of a subject prior to, during, and/or after initiation of chemotherapy and to protect and promote the regeneration of histological damage induced to host tissues by cytotoxic therapy to said subject.

In a second principal embodiment the invention provides a method of increasing bone marrow activity in a subject suffering myelosuppression as a consequence of cytotoxic therapy comprising administering to said subject an elsiglutide regimen before, during or after administering a cycle of said cytotoxic therapy to said subject.

In a third principal embodiment the invention provides a method of enhancing the effectiveness of cytotoxic therapy in a subject receiving cytotoxic therapy for the treatment of cancer comprising administering to said subject an elsiglutide regimen before, during or after administering a cycle of said cytotoxic therapy to said subject.

In various subembodiments of the first and second principal embodiments, said cytotoxic chemotherapy is characterized by a reduction in one or more hematological markers selected from the group consisting of white blood cell count, lymphocyte count, monocyte count, mean corpuscular volume, eosinophil count and mean corpuscular hemoglobin concentration, and said elsiglutide regimen causes a smaller reduction in said one or more hematological markers than would otherwise be observed in the absence of said elsiglutide administration.

In other subembodiments of the first and second principal embodiments, said subject is suffering from a reduction in one or more hematological markers selected from the group consisting of white blood cell count, lymphocyte count, monocyte count, mean corpuscular volume, eosinophil count and mean corpuscular hemoglobin concentration, and said elsiglutide regimen causes an increase in said one or more hematological markers.

In other subembodiments of the first and second principal embodiments, said cytotoxic chemotherapy is associated with one or more conditions selected from the group consisting of anemia (low red blood cell counts), neutropenia (low neutrophils counts), leucopenia (low white blood cell counts), and thrombocytopenia (low platelet counts), and said elsiglutide regimen treats said one or more conditions.

In still other subembodiments of the first and second principal embodiments, the subject is suffering from a condition selected from the group consisting of anemia (low red blood cell counts), neutropenia (low neutrophils counts), leucopenia (low white blood cell counts), thrombocytopenia (low platelet counts), and combinations thereof.

Still further independent embodiments provide:
A method of treating a subject suffering from or at risk for suffering a reduction in white blood cell count, optionally caused the administration of one or more cytotoxic agents, comprising administering to said subject a therapeutically effective amount of elsiglutide.
A method of treating a subject suffering from or at risk for suffering a reduction in lymphocyte count, optionally caused the administration of one or more cytotoxic agents, comprising administering to said subject a therapeutically effective amount of elsiglutide.
A method of treating a subject suffering from or at risk for suffering a reduction in monocyte count, optionally caused the administration of one or more cytotoxic agents, comprising administering to said subject a therapeutically effective amount of elsiglutide.
A method of treating a subject suffering from or at risk for suffering a reduction in mean corpuscular volume, optionally caused the administration of one or more cytotoxic agents, comprising administering to said subject a therapeutically effective amount of elsiglutide.
A method of treating a subject suffering from or at risk for suffering a reduction in eosinophil count, optionally caused the administration of one or more cytotoxic agents, comprising administering to said subject a therapeutically effective amount of elsiglutide.
A method of treating a subject suffering from or at risk for suffering a reduction in mean corpuscular hemoglobin concentration, optionally caused the administration of one or more cytotoxic agents, comprising administering to said subject a therapeutically effective amount of elsiglutide.
A method of treating a subject suffering from or at risk of suffering from anemia, optionally caused the administration of one or more cytotoxic agents, comprising administering to said subject a therapeutically effective amount of elsiglutide.
A method of treating a subject suffering from or at risk of suffering from neutropenia, optionally caused the administration of one or more cytotoxic agents, comprising administering to said subject a therapeutically effective amount of elsiglutide.
A method of treating a subject suffering from or at risk of suffering from leucopenia, optionally caused the administration of one or more cytotoxic agents, comprising administering to said subject a therapeutically effective amount of elsiglutide.
A method of treating a subject suffering from or at risk of suffering from thrombocytopenia, optionally caused the administration of one or more cytotoxic agents, comprising administering to said subject a therapeutically effective amount of elsiglutide.

In any of the foregoing embodiments, elsiglutide and a chemotherapeutic agent(s) are preferably administered concurrently for two or more days, with the elsiglutide administration beginning on the same day that the chemotherapy cycle begins, although it is feasible to administer or at least initiate the elsiglutide administration before the administration of the chemotherapeutic agent(s) begins, or to administer elsiglutide after the administration of the chemotherapeutic agent(s) concludes (i.e., during the days of the chemotherapy cycle when the chemotherapeutic agent(s) is no longer administered). When the chemotherapy comprises multiple cycles, such as 2, 3, 4 or more cycles, elsiglutide is preferably administered during each of the cycles. When administered on a daily basis, elsiglutide can be administered one or more times during the day, but it is preferably only administered once daily.

The elsiglutide regimen preferably comprises elsiglutide administration daily for 1, 2, 3, 4, 5, or six days of the chemotherapy cycle, or anywhere between these time periods (such as 1-5 days), although 4 days appears to be adequate. The regimen is also preferably initiated at the start of the chemotherapy cycle, although the regimen can also be initiated as many as 1, 2, 3, 4 or 5 days prior to the initiation of the chemotherapy cycle. The regimen is also preferably performed on consecutive days, although dosing for non-consecutive daily periods can also be envisioned.

A chemotherapy cycle may comprise administration of chemotherapy for 1 or more, 3 or more, 5 or more, 7 or more, 9 or more, or even 10 or more consecutive days during the cycle, or anywhere between these time periods (such as 1 to up to 5 days). The chemotherapy cycle might last for one week, two weeks, three weeks, four weeks, or even more, or anywhere in between these time periods. In one embodiment a limited period of elsiglutide administration is effective in the methods of the present invention throughout a 14 day chemotherapy cycle.

Any of the foregoing principal embodiments can be practiced with a wide range of chemotherapeutic agents. Non-limiting examples of such agents include anti-metabolites such as pyrimidine analogs (e.g., 5-fluorouracil [5-FU], floxuridine, capecitabine, gemcitabine and cytarabine) and purine analogs, folate antagonists and related inhibitors (e.g., mercaptopurine, thioguanine, pentostatin and 2-chlorodeoxyadenosine (cladribine)); antiproliferative/antimitotic agents including natural products such as vinca alkaloids (e.g., vinblastine, vincristine, and vinorelbine), microtubule disruptors such as taxanes (e.g., paclitaxel, docetaxel), vincristin, vinblastin, nocodazole, epothilones and navelbine, epidipodophyllotoxins (e.g., etoposide, teniposide), DNA damaging agents (e.g., actinomycin, amsacrine, anthracyclines, bleomycin, busulfan, camptothecin, carboplatin, chlorambucil, cisplatin, nedaplatin, cyclophosphamide, cytoxan, dactinomycin, daunorubicin, doxorubicin, epirubicin, aclarubicin, purarubicin, hexamethyhnelamineoxaliplatin, iphosphamide, melphalan, merchlorehtamine, mitomycin, mitoxantrone, nitrosourea, nimustine, ranimustine, estramustine, plicamycin, procarbazine, taxol, taxotere, teniposide, triethylenethiophosphoramide and etoposide (VP16)); antibiotics (e.g., dactinomycin (actinomycin D), daunorubicin, doxorubicin (adriamycin), idarubicin, anthracyclines, mitoxantrone, bleomycins, plicamycin (mithramycin), pleomycin, peplomycin, mitomycins (e.g., mitomycin C), actinomycins (e.g., actinomycin D), zinostatinstimalamer); enzymes (e.g., L-asparaginase); neocarzinostatin; antiplatelet agents; antiproliferative/antimitotic alkylating agents such as nitrogen mustards (e.g., mechlorethamine, cyclophosphamide and analogs, imidazol carboxamide, melphalan, chlorambucil, nitrogen mustard-N-oxide hydrochloride, ifosfamide), ethylenimines and methylmelamines (e.g., hexamethylmelamine, thiotepa, carboquone, triethylene thiophospharamide), alkyl sulfonates (e.g., busulfan, isoprosulfan tosylate), nitrosoureas (e.g., carmustine (BCNU) and analogs, streptozocin), trazenes-dacarbazinine (DTIC); epoxide type compounds (e.g., mitobronitol); antiproliferative/antimitotic antimetabolites such as folic acid analogs (e.g., methotrexate); platinum coordination complexes (e.g., cisplatin, carboplatin, oxaliplatin), procarbazine, hydroxyurea, mitotane, aminoglutethimide; hormones, hormone analogs (e.g., estrogen, tamoxifen, goserelin, bicalutamide, nilutamide) and aromatase inhibitors (e.g., letrozole, anastrozole); anticoagulants (e.g., heparin, synthetic heparin salts and other inhibitors of thrombin); fibrinolytic agents (e.g., tissue plasminogen activator, streptokinase and urokinase), aspirin, dipyridamole, ticlopidine, clopidogrel, abciximab; antimigratory agents; antisecretory agents (e.g., breveldin); immunosuppressives (e.g., cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blockers; nitric oxide donors; antisense oligonucleotides; antibodies (e.g., trastuzumab); cell cycle inhibitors and differentiation inducers (e.g., tretinoin); mTOR inhibitors, topoisomerase inhibitors (e.g., doxorubicin (adriamycin), amsacrine, camptothecin, daunorubicin, dactinomycin, eniposide, epirubicin, etoposide, idarubicin, mitoxantrone, topotecan, irinotecan); growth factor signal transduction kinase inhibitors; mitochondrial dysfunction inducers; chromatin disruptors; sobuzoxane; tretinoin; pentostatin; flutamide; porphimer natrium; fadrozole; procarbazine; aceglatone, and mitoxantrone. Other agents include monoclonal antibodies and other modalities that target vascular endothelial growth factor (VEGF) and its receptor (VEGFR) or epidermal growth factor (EGF), used alone and in combination with traditional small molecule chemotherapy. The method can also be practiced in conjunction with the administration of biologically targeted agents including but not limited to erlotinib, sorafenib, bevacizumzb, axitinib, sunitinib, and lapatinib.

The methods of the invention can be used in subjects suffering from a broad range of cancers, which subjects are subjected to anti-cancer chemotherapeutic treatments which result in deleterious side effects. Non-limiting examples of relevant cancers include, e.g., breast cancer, prostate cancer, multiple myeloma, transitional cell carcinoma, lung cancer (e.g., non-small cell lung cancer (NSCLC)), renal cancer, thyroid cancer and other cancers causing hyperparathyroidism, adenocarcinoma, leukemia (e.g., chronic myeloid leukemia, acute myeloid leukemia, chronic lymphocytic leukemia, acute lymphocytic leukemia), lymphoma (e.g., B cell lymphoma, T cell lymphoma, non-Hodgkins lymphoma, Hodgkins lymphoma), head and neck cancer, esophageal cancer, stomach cancer, colon cancer, intestinal cancer, colorectal cancer, rectal cancer, pancreatic cancer, liver cancer, cancer of the bile duct, cancer of the gall bladder, ovarian cancer, uterine endometrial cancer, vaginal cancer, cervical cancer, bladder cancer, neuroblastoma, sarcoma, osteosarcoma, malignant melanoma, squamous cell cancer, bone cancer, including both primary bone cancers (e.g., osteosarcoma, chondrosarcoma, Ewing's sarcoma, fibrosarcoma, malignant fibrous histiocytoma, adamantinoma, giant cell tumor, and chordoma) and secondary (metastatic) bone cancers, soft tissue sarcoma, basal cell carcinoma, angiosarcoma, hemangiosarcoma, myxosarcoma, liposarcoma, osteogenic sarcoma, angiosarcoma, endotheliosarcoma, lymphangiosarcoma, lymphangioendotheliosarcoma, synovioma, testicular cancer, uterine cancer, gastrointestinal cancer, mesothelioma, leiomyosarcoma, rhabdomyosarcoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, choriocarcinoma, seminoma, embryonal carcinoma, Wilms' tumor, epithelial carcinoma, glioma, glioblastoma, astrocytoma, medulloblastoma, craniopharyngioma, ependymoma, pinealoma, hemangioblastoma, acoustic neuroma, oligodendroglioma, meningioma, retinoblastoma, medullary carcinoma, thymoma, sarcoma, etc.

Specific elsiglutide doses useful in the methods of the invention will depend on the type of chemotherapy side effects to be treated, the severity and course of these side effects, previous therapy, the patient's clinical history and response to chemotherapy and elsiglutide, as well as the discretion of the attending physician. In one specific embodiment, such doses range from 5 to 80 or from 10 to 40 mg/day.

Specific non-limiting examples of useful routes of administration include subcutaneous, intravenous (IV), intraperitoneal (IP), and intramuscular.

In certain embodiments, elsiglutide is formulated in a pharmaceutical composition with a pharmaceutically acceptable carrier or excipient. In certain embodiments, elsiglutide is combined in a pharmaceutical composition together with another compound effective for ameliorating or preventing side effects of cancer chemotherapy. The formulations used in the methods of the invention may conveniently be presented in unit dosage form and may be prepared by methods known in the art. The amount of active ingredients that can be combined with a carrier material to produce a single dosage form will vary depending upon the host being treated and the particular mode of administration. The amount of active ingredients that can be combined with a carrier material to produce a single dosage form will generally be that amount of the compound which produces a therapeutic effect.

In general, the formulations can be prepared with a liquid carrier, or a finely divided solid carrier, or both, and then, if necessary, shaping the product. Pharmaceutical compositions suitable for parenteral administration may comprise elsiglutide in combination with one or more pharmaceutically acceptable sterile isotonic aqueous or non-aqueous solutions, dispersions, suspensions or emulsions, or sterile powders which may be reconstituted into sterile injectable solutions or dispersions just prior to use.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

Evaluation of the Effects of Elsiglutide Alone and in Combination with Irinotecan Chemotherapy on Hematopoiesis in Fischer Rats

A study was undertaken to evaluate the effects of elsiglutide alone and in combination with irinotecan chemotherapy on hematopoiesis in Fischer rats.

### Material and Methods

Animals.

8 to 12-week-old female Fischer 344/N rats (body weight 160-200 g) were obtained from Harlan Sprague Dawley Inc. (Indianapolis, Ind.).

Drugs and Formulation.

Irinotecan was purchased as a ready-to-use formulation solution at a concentration of 20 mg/ml (100 mg in a 5 ml vial). For a rat of 150-200 g, an administration up to 2 ml solution (dose of 200 mg/kg/d×3) was required.

Drug Doses and Schedule.

Elsiglutide was administered by subcutaneous (S.C) route at 1.8 mg/kg/day once a day for 4 days. Three doses were administered 30 minutes prior to each daily intravenous (I.V) dose of irinotecan. Only the fourth dose of elsiglutide was administered 24 hours after the last dose of irinotecan. Irinotecan was administered by intravenous (I.V) injection at the maximum tolerated and therapeutic dose, 100, 150, 200 mg/kg/day for 3 days.

Five rats were used for each experimental group, with several repeats for statistical significance

Necropsy.

The spleen and bone marrow of sternum of untreated Fischer rats and rats treated with irinotecan with 100, 150 and 200 mg/kg/d×3 alone and in combination with Elsiglutide (1.8 mg/kg/d×4) were examined histologically.

Hematological (CBC) Analysis
Complete blood cell count (CBC) analysis (days 0, 4, and 9)
From peripheral blood 14 markers, WBC (differential: neutrophils, lymphocytes, eosinophils, monocytes, basophils) and RDW-SD, RBC, HGB, HCT, MCV, MCH, MCHC, and Platelets were analyzed.

Histopathology.

Histologic analysis using sternum bone marrow and spleen were carried out on day 9 (6 days after the administration of the last irinotecan dose on day 3). Bone marrow and spleen specimens were fixed in buffered formalin (10%) for 48 hours. The entire sternum and spleen were removed during autopsy at the end of the experiments on day 9. The samples were put in 10% buffered formalin for 48 hours in order to get appropriate fixation. After that the sternum was decalcified in a Rapid Decalcifier (RDO) from Apex Engineering Products Corporation (Aurora, Ill., USA) for 17 minutes when the sternum could be bent easily with fingers. When decalcification was complete, samples were taken out of solution and put under cold running water for 60 minutes to remove the decalcification solution containing hydrochloric acid as an active ingredient. Then the decalcified bone marrow and spleen specimen were processed, embedded in paraffin, cut (5 u) and stained conventionally with hematoxylin and eosin (HE). The HE stained slides were used for general orientation and evaluation under light microscope using various magnifications.

Survival Experiments.

10 rats were assessed for survival up to 4 weeks after treatments. During the study, the kinetics of drug induced toxicities in animals (weight loss/gain, stomatitis, lethality were monitored daily during the first 2 weeks then twice a week thereafter up to 4 weeks in the survival phase. At the end of the survival phase the rats were sacrificed.

Statistical Analysis.

The statistical analysis of complete blood cells (CBC) results was carried out. The differences between the mean values in the different treatment groups were analyzed for significance.

Treatment groups and time when specific acquisition of tissues was obtained are outlined below. Fischer Rats (Female), weighing 150 to 180 g (8-12 weeks) were utilized.
Control, histology, d 9 and CBC, baseline, day 4 and day 9
Control vehicle, histology, d 9 and CBC baseline, d 4 and d 9
Irinotecan 100 mg/kg/d×3, I.V., histology, d 9 and CBC, baseline, d 4 and d 9
Irinotecan 150 mg/kg/d×3, I.V., histology, d 9 and CBC, baseline, d 4 and d 9, and CBC, baseline, d 4 and d 9
Irinotecan 200 mg/kg/d×3, I.V., histology 9, and CBC, d 4 and d 9
Elsiglutide 1.8 mg/kg/d×4, S.C., histology, d 9 and CBC, baseline, d 4 and d 9
CBC, baseline, d 4 and d 9
Elsiglutide 1.8 mg/d×4, S.C+Irinotecan 150 mg/kg/d×3, I.V, histology, d 9, and CBD baseline, d 4 and d9
Elsiglutide 1.8 mg/d×4, S.C.+Irinotecan 200 mg/kg/d×3, I.V, histology, d 9, and CBC d 4 and d 9
   Each treatment groups consisted of 10 rats for histology of bone marrow, spleen and peripheral blood analysis, and for survivors. Evaluation of peripheral blood was carried out on day zero (0), four (4) and day nine (9), bone marrow and spleen analysis after (necropsy) was carried out on day nine (9).

### Results

Analysis of the 14 biomarkers revealed that six biomarkers were significantly modified by Elsiglutide assessment on day 4 and 9 on surviving rats as outlined in Tables 1, 2, and 3. Fifty percent of rats treated with 200 mg/kg/d×3 and 10% of rats treated with the 150 mg/kg/d×3 irinotecan died because of toxicity on days 6 and 7. The analysis of the 6 biomarkers in the dead rats treated with irinotecan versus the animals which survived treatment with irinotecan alone (50%) and treated in combination with elsiglutide (100%), with no observable toxicity, revealed the protective effects of elsiglutide on the following six biomarkers: White Blood Cells (WBC), lymphocytes, monocytes, Mean Corpuscular Volume (MCV), eosinophil and Mean Corpuscular Hemoglobin Concentration (MCHC) as outlined in Tables 2 and 3. The 4 biomarkers which were not significantly modified by treatment in dead and alive rats include RBC, HGB, HCT, and MCH.

TABLE 1
Kinetics of modulation of blood markers altered by elsiglutide in rates treated with
a lethal dose of irinotecan 200 mg/kg/day × 3 days, assessment on days 4 and 9

| | | | Mean ± SD | Mean ± SD | P value | P value |
|---|---|---|---|---|---|---|
| Markers | Mean ± SD | Range | Day 4 | Day 9 | Day 4 | Day 9 |
| Neutrophils* | 0.95 ± 0.40 | 0.06-1.96 | 1.49 ± 0.07 | 1.03 ± 0.47 | <0.001 | NS |
| Platelets* | 48.74 ± 18.19 | 2.40-84.30 | 73.64 ± 10.66 | 58.34 ± 0.51 | <0.001 | NS |
| Basophils* | 1.15 ± 0.59 | 0.00-3.00 | 1.80 ± 0.84 | 0.2 ± 0.45 | NS | <0.01 |
| MCV* | 51.33 ± 1.98 | 49.00-57.5 | 51.00 ± 0.73 | 55.78 ± 1.58 | NS | 0.01 |
| MCHC* | 34.31 ± 1.19 | 31.30-36.10 | 34.88 ± 0.15 | 32.18 ± 0.83 | NS | <0.01 |
| RDW - SD* | 26.35 ± 1.81 | 23.90-32.50 | 25.98 ± 0.53 | 31.72 ± 4.45 | NS | <0.01 |

*Day 4 versus Day 9 are significant,
p < 0.01

TABLE 2
Markers significantly altered by elsiglutide, comparing dead rats with
Irinotecan on day 6 and on day 7(5 rats) versus rats alive on day 9
in irinotecan alone and in combination with elsiglutide (13 rats)

| | | | Mean Value ± SD | |
|---|---|---|---|---|
| | | Mean Value ± SD | (#Rats) | |
| | | (#Rats) | Irinotecan 200 + | |
| | | Irinotecan − 200 | Elsiglutide | |
| Markers | Control | (5 Dead) | (13 rats alive) | P value |
| WBC | 6.16 | 2.18 | 5.88 | <0.001 |
| Lymphocytes | 4.82 | 1.55 | 3.42 | <0.001 |
| Monocytes | 0.26 | 0.05 | 2.64 | <0.001 |
| MCV | 51.33 | 47.96 | 51.15 | <0.001 |
| Eosinophils | 0.12 | 0.42 | 0.03 | <0.001 |
| MCHC | 34.31 | 36.52 | 32.96 | <0.001 |

TABLE 3
Blood markers significantly altered in dead rats on day
6 and on day 7(5 rats) treatment with irinotecan (200 mg/kg/day ×
3 and rats survival on irinotecan alone and in combination
with Elsiglutide/Irinotecan day 9 (13 Rats).

| Markers | Groups | Permutation F-Test |
|---|---|---|
| WBC | Group 8 (I) Dead | P = <.001 |
| (Mean +/− 1 std) | Group 8 (I) Alive | |
| | Group 9 (E + I) Alive | |
| Lymphocytes | Group 8 (I) Dead | P = 0.003 |
| (Mean +/− 1 std) | Group 8 (I) Alive | |
| | Group 9 (E + I) Alive | |
| Monocytes | Group 8 (I) Dead | P = <.001 |
| (Mean +/− 1 std) | Group 8 (I) Alive | |
| | Group 9 (E + I) Alive | |
| MCV | Group 8 (I) Dead | P = 0.002 |
| (Mean +/− 1 std) | Group 8 (I) Alive | |
| | Group 9 (E + I) Alive | |
| Eosinophils | Group 8 (I) Dead | P = 0.007 |
| (Mean +/− 1 std) | Group 8 (I) Alive | |
| | Group 9 (E + I) Alive | |
| MCHC | Group 8 (I) Dead | P = <.001 |
| (Mean +/− 1 std) | Group 8 (I) Alive | |
| | Group 9 (E + I) Alive | |

### Example 2

Histopathological Evaluation of Bone Marrow of Rat Sternum and Spleen after Treatments with Elsiglutide, Irinotecan Alone and with their Combination

Another study was undertaken to determine the potential of elsiglutide in reversal of bone marrow toxicity induced by irinotecan. Regulatory guidelines and recommendations according to the published literature were followed during the evaluation of bone marrow (Reagen W J et al, TOXICOLOGIC PATHOLOGY, 39:435-448, 2011).

### Results of Histopathological Evaluation of Sternum Bone Marrow and Spleen (5 Rats Per Group.)

a. Untreated Controls and Vehicle Treated (Groups 1-2)

Bone marrow of sternum showed normal histological structure. In these female Fisher-rats it is part of the normal histological structure that the available space for hematopoiesis is not completely utilized (unlike bone marrow of Swiss mice) because of the presence of adipose (fat) tissue what characteristically always infiltrates the bone marrow overall in about 30%. The area in bone marrow covered by fat tissue was pretty consistent and did not show significant individual differences. In untreated animals, the hematopoietic tissue area was about 70% of the entire bone marrow territory and the rest of 30% was adipose tissue. The spleen showed normal histological structure. The red pulp contained scattered, single, matured megakaryocytes in average 8 (range 5-10) in the entire longitudinal cross-section of the spleen. No extra medullary hematopoietic foci were seen in the red pulp.

b. Elsiglutide 1.8 mg/kg Treatment (Group 3)

Bone marrow from four animals showed normal histological structure with about 30% adipose tissue content. Histological architecture of spleens were normal. Average number of megakaryocytes was 10 (range 6-15) which cannot be considered significantly different compared to the numbers of megakaryocytes seen in spleens (average 8, range 5-10) from untreated rats.

c. Irinotecan 100 mg/kg i.v. Treatment (Group 4)

Two animals showed normal histology and overall ratio of hematopoietic tissue (70%) and fat tissue (30%). In 3 rats slightly increased cellularity in bone marrow, and decreased amount of fat tissue (10-20%) was seen in bone marrow sections. The spleen from all 5 animals showed the presence of extra medullary hematopoietic foci in the red pulp in numerous locations. These hematopoietic cells were immature without the presence of matured segmented granulocytes. The average number of megakaryocytes increased to 23 (range 10-60). The megakaryocytes appeared as multinucleated giant cells containing multiple, large round-shaped nuclei characteristic for immature megakaryocytes as compared to the matured megakaryocytes with darkly-stained nuclei of shrunken chromatin seen in normal rat spleen.

d. Irinotecan 150 mg/kg i.v. Treatment (Group 5)

One rat died on day 8 with decreased bone marrow cellularity, increased amount of adipose tissue (70%). The bone marrow contained a small necrotic area, hemorrhages indicative of myelotoxicity with about 40% myelosuppression. I.e., about 40% of the total bone marrow was destroyed and not involved in hematopoiesis. In the spleen, the red pulp contained large areas of hemosiderin and hemosiderin containing macrophages. Hemosiderin is a breakdown product of erythrocytes. Extra medullary hematopoietic foci was not seen in the red pulp of the spleen, indicating that irinotecan could have inhibited the compensatory, backup hematopoiesis in the spleen after inducing myelotoxicity in bone marrow. Another animal was sacrificed on day 8 in moribund state. The slide from spleen showed hematopoietic foci and significant hemosiderin containing macrophages in the pulp indicative (but in this case not proven) of previous myelotoxicity caused by irinotecan on days 1-3. Increased cellularity with significant megakaryocytes, decreased fat tissue content (20%), and no sign of necrosis was seen in the bone marrow of 2 other rats. The spleen showed significant medullary hematopoietic foci, with an increased number (1625) of megakaryocytes. These findings in bone marrow and spleen on day 9 show reactive spontaneous compensatory bone marrow regeneration, as a consequence of earlier bone marrow toxicity caused by irinotecan on days 1-3. One animal showed normal histological structures in the bone marrow and in the spleen.

e. Irinotecan 100 mg/kg i.v with Elsiglutide (Group 6)

In this combination treatment group, the bone marrow of 3 animals showed normal histological structure. In 2 animals, increased cellularity with significant megakaryocytes and decreased fat tissue (˜10%) was seen. In the spleens, extensive hematopoiesis was observed almost in the entire red pulp with a significant number of megakaryocytes (range=25-90) in 4 rats.

f. Irinotecan 150 mg/kg i.v. with Elsiglutide (Group 7)

No death or moribund condition occurred in this group. Normal histological structure of bone marrow was seen in 3 of 5 rats. Increased cellularity with significant numbers of megakaryocytes and decreased adipose tissue content (˜10-20%) was seen in the bone marrow of 2 of 5 rats. One spleen exhibited normal histological structure; in the other 4 spleens several hematopoietic foci were seen in the red pulp, demonstrating that elsiglutide prevented the development of 1 death with 40% myelosuppression in the bone marrow and 1 moribund condition with very probable myelosuppression what was the key finding in the group 5 treated with irinotecan 150 mg/kg alone.

g. Irinotecan 200 mg/kg i.v. Treatment (Group 8)

One animal died with severe diarrhea on day 7. Another 4 rats were in moribund condition with acute diarrhea and they were sacrificed before day 9 after the full treatment. This saved the bone marrow and spleen for reliable histological investigation without the tissue damage which could have been caused by autolysis if death was delayed. The histology of bone marrow and spleen of the 5 dead animals were very similar. Severe myelosupression (myelotoxicity) was caused by 2×MTD dose of irinotecan. The characteristic feature of the sternum bone marrow was the 95-98% decrease (myelosuppression) in hematopoiesis. It was evidenced by the very low level of hematopoietic cellularity. Few lymphocytes, blast cells (stem cells) nuclear fragments remained in the increased adipose tissue which basically replaced the real hematopoetic tissue. Various sizes of microscopic hemorrhages were seen in the almost empty bone marrow. The spleens of these animals did not show any sign of extramedullary hematopoiesis. Significant hemosiderin and hemosiderin containing macrophages were seen in the red pulp of the spleen. The surviving 5 other animals were autopsied on day 9 as scheduled. In these animals, the bone marrow was regenerated by that time. It was normal or hypercellular (indicating the regeneration) with the decrease of fat tissue. But the spleens showed a characteristic extramedullary hematopoiesis evidenced by several hematopoietic foci mixed with red blood cells in the pulp. This is a compensatory effect to the previous myelotoxicity caused by irinotecan, still present on day 9.

h. Irinotecan 200 mg/kg i.v. Treatment with Elsiglutide (Group 9)

One of 10 animals died on day 7 with diarrhea. Histology of bone marrow and spleen was very similar in these animals. The bone marrow showed very low cellularity (20%) and the area of adipose tissue increased (80%). Multiple microscopic hemorrhages were also seen. The estimated real myelosuppression was about 80% in these animals. No hematopoiesis was seen in the spleens of these animals. The surviving 8 other animals were autopsied on day 9 as scheduled. The bone marrow of all 8 animals showed normal histology: 70% myelopoietic tissue and 30% adipose tissue. The cellular composition of the myelopoiesis did not show any abnormality. But the spleen contained several foci of extramedullary hematopoietic foci mixed with red blood cells in the pulp. This is a compensatory mechanism in the spleen of rats to previous myelotoxicity and it is still visible on day 9.

Summary of WBC/RBC/Platelet/Group

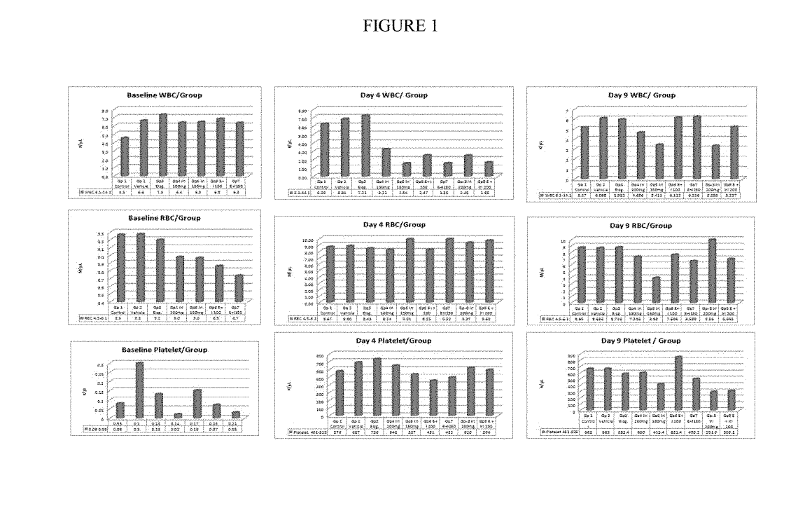

FIG. 1 demonstrates the changes of the average numbers of the major peripheral blood cell types (WBC, RBC, Platelets) in the various groups on Day 4 and Day 9. It shows that on Day 4: WBC/group decreased after each dose of irinotecan. RBC/Group and Platelet/Group did not change. Elsiglutide did not prevent the development of neutropenia indicated by the bone marrow damage present in the combination group. On Day 9 Elsiglutide increased the WBC/Group count to normal level in the combination groups. The WBC/Group in the irinotecan alone treated group showed an increase because of spontaneous regeneration but did not reach the normal level. The Platelet/Group count decreased after 200 mg/kg irinotecan and did not reach the normal level with Elsiglutide combination by day 9.

### Example 3

Elsiglutide Enhances Therapeutic Response to Chemotherapy and Provides Selective Protection Against Organ-Specific Toxicities Induced by Chemotherapeutic Agents in Mice and Rats.

Studies were carried out in normal rats, rats bearing colon tumors and xenografts bearing human colon carcinoma, HCT8 and HT-29. Studies were carried out to test the hypothesis that elsiglutide offers selective protection against 5-Fluorouvacil-(5-FU) and irinotecan-induced toxicities and potentially enhances their antitumor activity. Elsiglutide was administered subcutaneously at non-toxic but therapeutically-effective dose of 1.8 mg/kg/day daily for 4 days, 30 min prior to each 5-FU/irinotecan dose. The tested doses of 5-FU were 100 mg/kg (MTD) and 200 mg/kg, and the doses of irinotecan were 100 mg/kg (MTD) and 200 mg/kg, either daily ×3 or weekly ×4.

The results generated indicate that elsiglutide offers selective protection against 5-FU-/irinotecan. The histological damage induced by lethal dose of 5-FU and irinotecan was restored by elsiglutide with no significant effects on proliferation index of normal and tumor tissues. Furthermore, in tumor-bearing rodents, the kinetics of response observed with 5-FU and irinotecan were enhanced by several days and the overall tumor growth inhibition increased from 30% (with 5-FU/irinotecan alone) to 80% upon the administration of elsiglutide. Collectively, these results demonstrate that elsiglutide offers selective protection against 5-FU- and irinotecan-induced organ-specific toxicity, and offers the potential for enhanced therapeutic response.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A method of increasing bone marrow activity in a subject suffering myelosuppression as a consequence of cytotoxic therapy comprising administering to said subject an elsiglutide regimen before, during or after administering a cycle of said cytotoxic therapy to said subject.

2. A method of improving the immunological status of a subject immune-compromised as a consequence of cytotoxic therapy comprising administering to said subject an elsiglutide regimen before, during or after administering a cycle of said cytotoxic therapy to said subject.

3. A method of enhancing the effectiveness of cytotoxic therapy in a subject receiving cytotoxic therapy for the treatment of cancer comprising administering to said subject an elsiglutide regimen before, during or after administering a cycle of said cytotoxic therapy to said subject.

4. The method of claim 1, wherein said cytotoxic chemotherapy is characterized by a reduction in one or more hematological markers selected from the group consisting of white blood cell count, lymphocyte count, monocyte count, mean corpuscular volume, eosinophil count and mean corpuscular hemoglobin concentration, and said elsiglutide regimen causes a smaller reduction in said one or more hematological markers than would otherwise be observed in the absence of said elsiglutide administration.

5. The method of claim 1, wherein said subject is suffering from a reduction in one or more hematological markers selected from the group consisting of white blood cell count, lymphocyte count, monocyte count, mean corpuscular volume, eosinophil count and mean corpuscular hemoglobin concentration, and said elsiglutide regimen causes an increase in said one or more hematological markers.

6. The method of claim 1, wherein said cytotoxic chemotherapy is associated with one or more conditions selected from selected from the group consisting of anemia (low red blood cell counts), neutropenia (low neutrophils counts), leucopenia (low white blood cell counts), and thrombocytopenia (low platelet counts), and said elsiglutide regimen treats said one or more conditions.

7. The method of claim 1, wherein the subject is suffering from a condition selected from the group consisting of anemia (low red blood cell counts), neutropenia (low neutrophils counts), leucopenia (low white blood cell counts), thrombocytopenia (low platelet counts), and combinations thereof.

8. The method of claim 1, wherein said elsiglutide regimen comprises daily administration of elsiglutide for 2 to 6 days, and said cycle of cytotoxic therapy is 8 to 24 days.

9. The method of claim 1, wherein said elsiglutide regimen comprises daily administration of elsiglutide for a plurality of consecutive days from the beginning of the cycle of cytotoxic therapy.

10. The method of claim 1, wherein the elsiglutide is administered during at least the first two consecutive days from the beginning of the cycle of cytotoxic therapy.

11. The method of claim 1, wherein the elsiglutide is administered during at least the first four consecutive days from the beginning of the cycle of cytotoxic therapy.

12. The method of claim 1, wherein the elsiglutide is administered for two cycles of cytotoxic therapy during the first four consecutive days from the beginning of each cycle of cytotoxic therapy.

13. The method of claim 1, wherein the cycle of cytotoxic therapy is up to 14 days long.

14. The method of claim 1, wherein the cycle of cytotoxic therapy is 14 days or longer.

15. The method of claim 1, wherein the elsiglutide regimen comprises a therapeutically effective amount of elsiglutide of about 10-40 mg/day.

16. The method of claim 1, wherein the elsiglutide regimen comprises a therapeutically effective amount of elsiglutide selected from about 10 mg/day, about 20 mg/day, and about 40 mg/day.

17. The method of claim 1, wherein the cytotoxic therapy comprises administration of one or more compounds selected from the group consisting of antimetabolites, alkylating agents, anticancer antibiotics, microtubule-targeting agents, topoisomerase inhibitors, alkaloids, antibodies, pyrimidine analogs, purine analogs, folate antagonists, epidipodophyllotoxins, DNA damaging agents, antiplatelet agents, platinum coordination complexes, hormones, hormone analogs, aromatase inhibitors, anti-angiogenic compounds, growth factor inhibitors, angiotensin receptor blockers, nitric oxide donors, antisense oligonucleotides, cell cycle inhibitors, differentiation inducers, mTOR inhibitors, mitochondrial dysfunction inducers, chromatin disruptors.

18. The method of claim 1, wherein the cytotoxic therapy comprises administration of one or more compounds selected from the group consisting of 5-fluorouracil (5-FU), floxuridine, capecitabine, gemcitabine, cytarabine, irinotecan, doxorubicin (adriamycin), amsacrine, camptothecin, daunorubicin, dactinomycin, eniposide, epirubicin, etoposide, idarubicin, mitoxantrone, topotecan, oxaliplatin, cisplatin, carboplatin, folinic acid, methotrexate, and biologically targeted agents selected from erlotinib, sorafenib, bevacizumzb, axitinib, sunitinib, and lapatinib.

19. The method of claim 1, wherein the cytotoxic therapy comprises administration of 5-fluorouracil or irinotecan.

20. The method of claim 1, wherein the cytotoxic therapy is administered as a FOLFOX or FOLFIRI cytotoxic therapy regimen.

21. The method of claim 1, wherein the elsiglutide is administered subcutaneously (s.c.).

22. The method of claim 1, wherein the elsiglutide is administered intravenously or intraperitoneally.

23. The method of claim 1, wherein the subject is a human.

24. The method of claim 1, wherein the subject has a cancer with performance status of ≤2 according to the Eastern Cooperative Oncology Group (ECOG).

25. The method of claim 1, wherein the subject is cytotoxic therapy-naïve prior to the start of the first cycle of cytotoxic therapy.


SEQUENCE LISTING (2 sequences)

SEQ ID NO: 1 - protein, 39 aa, artificial sequence
  elsiglutide chemically synthesized
HGEGSFSSELSTILDALAARDFIAWLIATKITDKKKKKK

SEQ ID NO: 2 - protein, 33 aa, human
HADGSFSDEMNTILDNLAARDFINWLIQTKITD